(19) United States
(12) Patent Application Publication
(10) Pub. No.: US 2020/0138199 A1
(43) Pub. Date: May 7, 2020

(54) SYSTEM AND METHOD FOR DELIVERING VIBROACOUSTIC STIMULATION

(71) Applicant: Holistic Health Science, LLC, San Diego, CA (US)

(72) Inventor: Dominic Carnevale, San Diego, CA (US)

(21) Appl. No.: 16/670,706

(22) Filed: Oct. 31, 2019

Related U.S. Application Data
(60) Provisional application No. 62/754,337, filed on Nov. 1, 2018.

(51) Int. Cl. A47C 15/00; A47C 3/16; A47C 7/72; A61M 21/02

(52) U.S. Cl. CPC A47C 15/004; A47C 3/16; A61M 2021/0022; A61M 21/02; A47C 7/727

(57) ABSTRACT

This disclosure provides systems, methods, and devices for delivering vibroacoustic stimulation for enhancing a meditation experience. The system can include a seating apparatus. The seating apparatus can be a piece of furniture such as meditation cushion, a chair, mat, or pad, or the system can be embodied within a block or similar. The system can receive an audio signal via wired or wireless means. The system can produce a vibroacoustic output via one or more tactile transducers that product vibrations according to low frequency components of the audio signal. The system can also have one or more listening devices for producing an audible sound according to the audio input. The listening devices can be speakers or headphones coupled to the system via a wired or wireless link.

### CROSS-REFERENCE TO RELATED APPLICATIONS

This application claims priority to U.S. Provisional Patent Application Ser. No. 62/754,337, filed Nov. 1, 2018, entitled, “SYSTEM AND METHOD FOR DELIVERING VIBROTACTILE THERAPY,” the contents of which are hereby incorporated by reference in their entirety.

### BACKGROUND

### Technical Field

This disclosure relates to vibrotactile or vibroacoustic technologies. More specifically, this disclosure relates to systems and methods for delivering vibroacoustic stimulation at natural frequencies in a passive and non-invasive manner.

### Related Art

Listening to music can promote a subjective feeling of wellbeing and induce a psychophysiological relaxation response, characterized by a reduction of the sympathetic activity of the autonomic nervous system. Some clinical applications of musical intervention have induced reductions of heart rate, blood pressure and respiratory rate, as well as decreases in anxiety and improvements of mood in different patient groups. Especially slow and soothing music seems to facilitate the vegetative relaxation effect. Vibrations applied to portions of the body in conjunction with such musical interventions can further increase relaxation and enhance meditation and mindfulness exercises.

### SUMMARY

One aspect of the disclosure provides a system for delivering vibroacoustic stimulation. The system can include a seating apparatus. The system can include an acoustic subsystem contained within the seating apparatus having an audio input configured to receive an audio signal. The system can include one or more tactile transducers contained within the seating apparatus and communicatively coupled to the acoustic subsystem and configured to provide vibrations according to low frequency elements of the audio signal. The system can include a listening device coupled to the acoustic subsystem and configured to produce an audio signal to a user in conjunction with the vibrations. The system can include a user interface configured to receive user input regarding the audio signal.

Another aspect of the disclosure provides a device for enhanced meditation experience. The device can include a meditation cushion having an upper portion for receiving a rear of a user and a lower portion for receiving feet and legs of the user. The device can include an audio input configured to receive an audio signal. The device can include one or more tactile transducers contained within the meditation cushion and communicatively coupled to the acoustic subsystem, the one or more tactile transducers being configured to provide vibrations according to low frequency elements of the audio signal. The device can include a listening device coupled to the acoustic subsystem and configured to produce an audio signal to a user in conjunction with the vibrations. The device can include a user interface configured to receive user input regarding the audio signal.

Another aspect of the disclosure provides a device providing vibroacoustic stimulation. The device can have a rigid body having rectangular prism shape. The device can have an audio input configured to receive an audio signal. The device can have at least one tactile transducer disposed within the rigid body and configured to product vibrations according to low frequency components of the audio signal. The device can have a listening device configured to output an audible version of the audio signal synchronized with the vibrations.

The rigid body can be a block. The audio input can include a first wireless link. The audio output can include a second wireless link.

Other features and advantages will be apparent to one of skill in the art with a review of the detailed description.

### BRIEF DESCRIPTION OF THE FIGURES

The details of embodiments of the present disclosure, both as to their structure and operation, may be gleaned in part by study of the accompanying drawings, in which like reference numerals refer to like parts, and in which:

FIG. 1 is a graphical depiction of an embodiment of a vibroacoustic stimulation delivery system;

FIG. 2 is a functional block diagram of the vibroacoustic stimulation delivery system of FIG. 1; and

FIG. 3 a top perspective view of an embodiment of the vibroacoustic stimulation delivery system of FIG. 1;

FIG. 4 is a front elevation view of the vibroacoustic stimulation delivery system of FIG. 3;

FIG. 5 is a rear elevation view of the vibroacoustic stimulation delivery system of FIG. 3;

FIG. 6 is a right-side elevation view of the vibroacoustic stimulation delivery system of FIG. 3;

FIG. 7 is a left-side elevation view of the vibroacoustic stimulation delivery system of FIG. 3;

FIG. 8 is a plan view of the vibroacoustic stimulation delivery system of FIG. 3;

FIG. 9 is a bottom view of an embodiment of the vibroacoustic stimulation delivery system of FIG. 1;

FIG. 10 is a bottom view of another embodiment of the vibroacoustic stimulation delivery system of FIG. 1;

FIG. 11 is a graphical representation of another embodiment of the vibroacoustic stimulation delivery system of FIG. 1;

FIG. 12 is a graphical representation of another embodiment of the vibroacoustic stimulation delivery system of FIG. 1; and

FIG. 13 is a graphical representation of another embodiment of the vibroacoustic stimulation delivery system of FIG. 1.

### DETAILED DESCRIPTION

A system for creating a space for meditation and mindfulness is provided. The system can be formed from a cushion adapted to receive a seated person. The system can include an audio subsystem configured to deliver vibrations throughout the cushion to enhance the human experience of sound and further optimize active meditation, relaxation, therapy, learning and enjoyment of sound. This is referred to herein as vibro-acoustic or vibroacoustic stimulation, creating physical stimulation to the human body using sound. The audio subsystem can further deliver an audio signal to one or more speakers, headphones, etc.

The detailed description set forth below, in connection with the accompanying drawings, is intended as a description of various embodiments and is not intended to represent the only embodiments in which the disclosure may be practiced. The detailed description includes specific details for the purpose of providing a thorough understanding of the embodiments. However, it will be apparent to those skilled in the art that the disclosure without these specific details. In some instances, well-known structures and components are shown in simplified form for brevity of description.

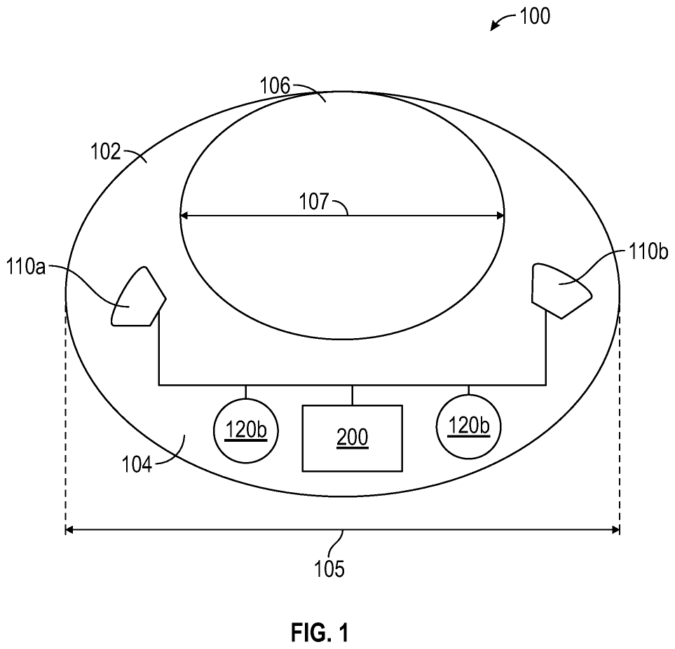

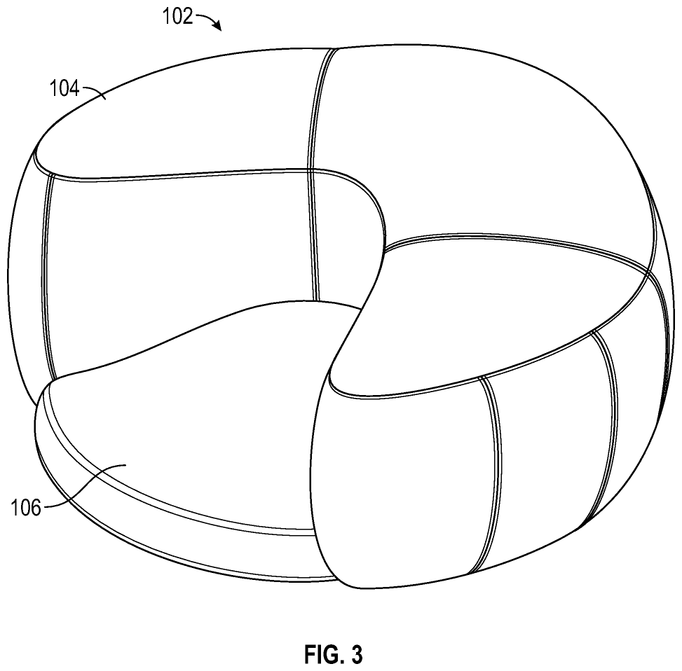

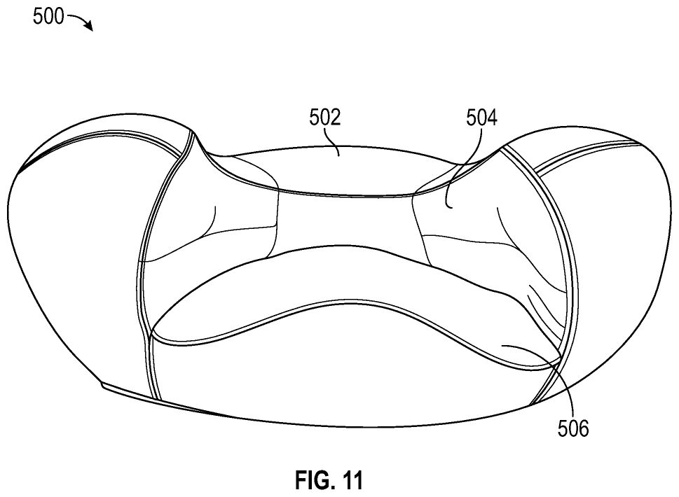

FIG. 1 is a graphical depiction of an embodiment of a vibroacoustic stimulation delivery system. A vibroacoustic stimulation delivery system (system) 100 can have a seating apparatus 102. The seating apparatus 102 can be a piece of furniture, such as a bed, chair, a couch, a sitting block, a table, a matt, a stool or a sitting cushion. The seating apparatus 102 can be formed as an ergonomic cushion, seat, or other apparatus or platform that can be used for meditation (e.g., a meditation cushion). The seating apparatus 102 can have an upper portion 104. The upper portion 104 can be, for example a sitting area or an area against which a user can lean. The seating apparatus 102 can also have a lower portion 106. The lower portion 106 can receive the feet of the user in for example, a crossed-legged sitting style or position (otherwise known as Lotus or Indian Style seating position). The embodiment shown in FIG. 3 through FIG. 8 depicts the two-tiered construction allowing the feet and lower legs to be received in the lower portion 106 while the user can sit on the upper portion 104. Additional embodiments of the system 100 are shown in FIG. 11 through FIG. 13.

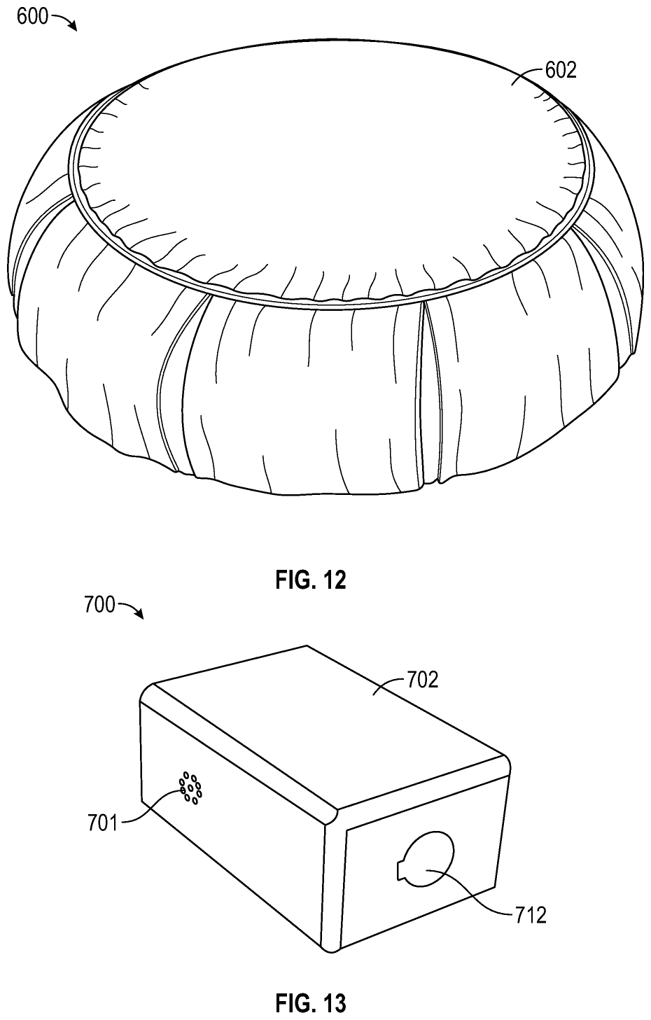

In some embodiments, the seating apparatus 102 can be round (see FIG. 12), oval, square or have a rounded-triangular profile (see FIG. 11), for example. In some implementations having a multiple section, round construction as those in FIG. 3 through FIG. 11, the upper portion 104 and the lower portion 106 can have a radius or diameter related by the Golden Ratio or 1.61 (1.61803398875). For example, a diameter 105 of the upper portion 104 can be 1.618 times a diameter 107 of the lower portion 106.

In some implementations such as that shown in FIG. 12, the upper portion 104 and the lower portion 106 may not be separately identified. Thus, as described below, the system 100 can be included in a single-piece cushion or mat.

The system 100, or the seating apparatus 102 more specifically, can have an ergonomic, decorative, or simple and portable construction. The system 100 can have a control system 200. The control system 200 can be communicatively coupled to one or more listening devices 110 (shown as a listening device 110a and a listening device 110b). The listening devices 110 can include a set of headphones worn by a user. The listening devices 110 can include sound-emitting equipment embedded within the seating apparatus 102, and/or contained within internal compartments within the upper portion 104 and/or the lower portion 106. In some implementations, the upper portion 104 may contain a majority of the electronic components. The listening devices 110 can include both a set of headphones worn by the user and speakers embedded within the seating apparatus 102 as desired. In some implementations, the listening devices 110 can also include external standalone speakers such as, a bookshelf speaker or a tower speaker. One, three, four, or even five or more listening devices 110 can be included for use with the system 100. Several implementations of the listening devices 110 are possible and are readily combinable with each other. For example, internal speakers may be present in addition to headphones.

The control system 200 can also be coupled to one or more tactile transducers 120 (shown as tactile transducers 120a, 120b). The one or more tactile transducers 120 can provide vibrations synchronized with corresponding sound from an audio file, for example, emitted from the listening devices 110. The synchronized sound from the listening devices 110 and vibrations from the tactile transducers can be used to deliver vibroacoustic stimulation or create an enhanced meditation environment for the user. The tactile transducer 120 can be a vibrotactile transducer configured to emit vibrations corresponding to, for example, music/audio or a recording played by the vibroacoustic stimulation delivery system 100. The vibrations can therefore be consistent with a melody or bass of a song, for example. The vibrations can generally correlate to various low-frequency aspects of a given song or recording. In some implementations, low-frequency in this sense can vary from 5 hertz (Hz) to 17 kHz. In some implementations, the low-frequency aspects can include transmissions below 5 Hz.

As used herein, the tactile transducer 120 (or vibrotactile transducer) is an electro-mechanical device that shakes, vibrates, or resonates based on an electrical signal such as an audio signal. The tactile transducer 120 can be compared to a loudspeaker or subwoofer driver minus the cone/diaphragm. It can include a damped mass on a spring driven by an oscillating force. The tactile transducers 120 can turn the lower frequency components of an audio signal into vibrations transmitted through the seating apparatus 102.

In some embodiments, the control system 200, the tactile transducer(s) 120, and the listening devices 110 can be embedded or otherwise contained within the seating apparatus 102. In some embodiments, a control system 200 and associated audio and vibration components can be used in any suitable structure such as a bed, a couch, a block a matt, a table, a chair, or other suitable seating apparatus or furniture. As such, this disclosure is not limited to the ergonomic seating apparatus 102.

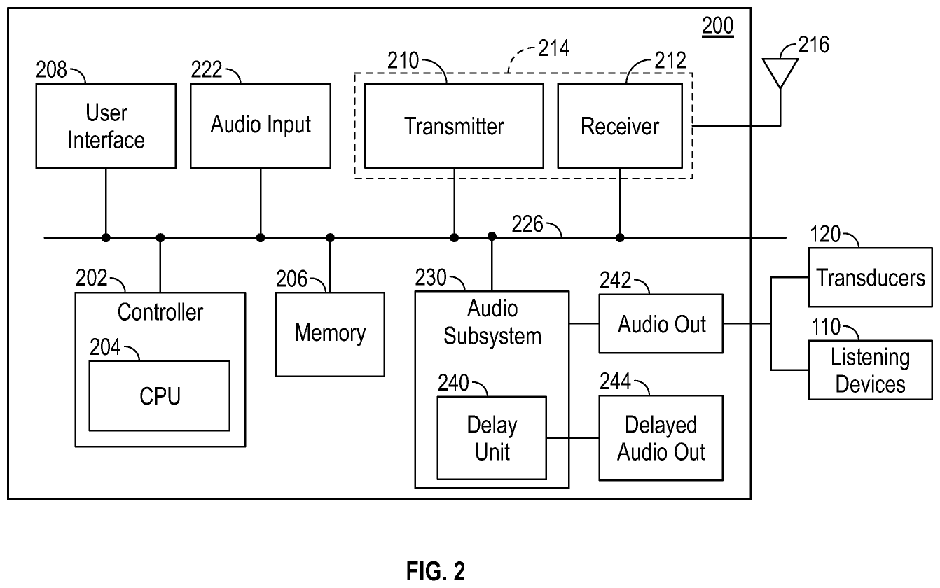

FIG. 2 is a functional block diagram of an embodiment of the vibrotactile delivery system of FIG. 1. The control system 200 can have a controller 202. The controller 202 can further include one or more processors or microprocessors implemented as a central processing unit (CPU) 204. The controller 202 can be coupled to a memory 206. The memory 206 can have one or more memory units configured to store executable instructions for the CPU 204 memory 206 can store programming instructions for the music playback and vibration delivery according to an audio input (e.g., the audio input 222, below).

The controller 202 can be coupled to a user interface 208. The user interface 208 can have one or more components used to receive user inputs and adjust audio and vibratory outputs. The user interface can also have display system output or control output of the control system 200. The user interface 208 can include knobs, dials, buttons, an electronic peripheral connection port (e.g., Universal Serial Bus (USB) port), a keyboard, a mouse, a touchscreen, or other similar modules, parts, or elements that allow a user to interact with the system 200 directly or via a remote control (e.g., a smartphone).

The controller 202 can be communicatively coupled to an audio input 222. The audio input 222 can receive input (e.g., audio input, media files, or sound) via a wired or wireless connection. The input can be audio, video, or multimedia files of various formats. For example, the audio input can be MP3, .wav, .m4a, or other suitable format. The audio input 222 can also receive such input signals via wireless protocols, such as Bluetooth, Zigbee, and the IEEE 802.XX family of protocols (e.g., Wi-Fi), for example.

The controller 202 can be communicatively coupled to a transmitter 210. The controller can be communicatively coupled to a receiver 212. The transmitter 210 and the receiver 212 can be implemented together as a transceiver 214. The transmitter 210 and the receiver 212 or the transceiver 214 can transmit and receive wireless signals via one or more antennas 216. In some implementations, the transceiver 214 or receiver 212 can receive, for example, wireless transmissions for the audio input 222 from a user device such as a smartphone or other mobile wireless device. In some examples, the user device can also function as a portion of the user interface 208 via the transceiver 214.

In some other implementations, the transceiver 214 or the transmitter 210 can transmit information, data, or other audio signals to an external source. For example, the user can use wireless headphones or a wireless speaker to listen to music or other recordings while using the system 100 (e.g., seated on the seating apparatus 102). In some implementations, the transceiver 214 can include one or more transceivers (e.g., Bluetooth transceivers). For example, a first transceiver can be used to receive streaming audio from a user device (e.g., a smartphone) while a second can transmit the media or recordings to the wireless headphones.

The controller 202 can be coupled to an audio subsystem 230. The audio subsystem 230 can have one or more amplifiers (e.g., low noise amplifiers, power amplifiers), processors configured to convert audio files or music received at the audio input 222 as needed for output. The audio subsystem 230 can provide music to the listening devices 110 via an audio output 242. Audio can also be delivered to another external audio playback system. The external audio playback system can be directly coupled to the audio output 242 or wirelessly coupled to the audio output 242 via the transceiver 214, for example. The audio subsystem 230 can provide input to the tactile transducer(s) 120 via the audio output 242. The tactile transducer(s) 120 may only replicate low-frequency tones, as noted above. The frequency band(s) reproduced by the tactile transducers 120 can be adjusted manually via, for example, the user interface 208.

The audio subsystem 230 can have a delay unit 240. The delay unit 240 can provide an integrated, selectable or automatic delay function and preamp for control of audio provided to a delayed audio output 244. In some examples, the vibrations at the tactile transducer 120 may be delayed from the audio at the audio output 242. This is because the transducer 120 forms in-air vibrations from an audio file or music received at the audio input 222. The vibrations produced by the transducer and felt by the user take longer to reach the user than the audio directly output to the listening devices 110. Thus, the vibrations may be delayed (in time and/or phase) from the corresponding music. This delay causes a latency between the audio heard by the user and associated vibrations felt by the user. This presents a sub-optimal meditative experience because sound from the audio output 242 is not synchronized with the vibrations caused by the transducer 120 according to the music/bass. The delay unit 240 can allow a user to customize or otherwise adjust the amount of delay as needed to ensure proper synchrony between sounds and vibrations.

The delay unit 240 can further have a power amplifier for further amplification and to compensate for any loss in transmission. The delayed audio output 244 can be coupled to, for example, the set of headphones worn by a user. This can include wired headphones or wireless headphones (e.g., via the transceiver 214), or other suitable listening devices (e.g., the listening devices 110). The delay unit 240 can provide a user-selectable delay to adjust a timing of the music heard via the delayed audio output 244 and to synchronize it with the vibrations felt from the transducer 120. In some embodiments, the delay unit 240 can provide a user-adjustable delay on the order of milliseconds. In some embodiments, the delay can be automatically set by the controller 202, for example. In some embodiments, the delay can be adjusted in increments from 30 milliseconds (ms) to 360 ms of delay.

The delay unit 240 may be optional and may not be present in every embodiment of the vibroacoustic stimulation delivery system 100.

FIG. 3 a top perspective view of an embodiment of the vibroacoustic stimulation delivery system of FIG. 1.

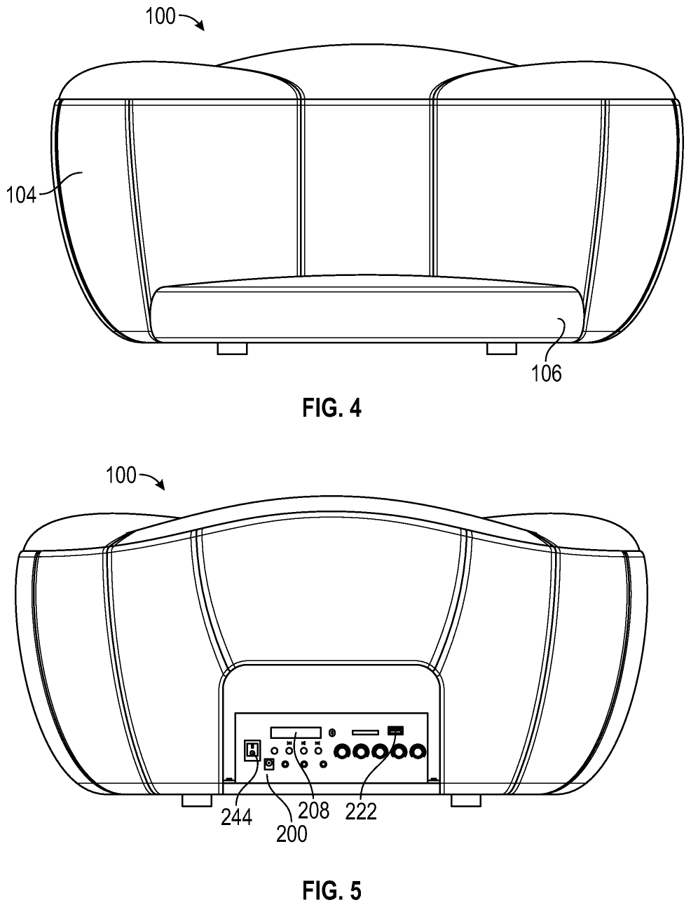

FIG. 4 is a front elevation view of the vibroacoustic stimulation delivery system of FIG. 3. A user can sit on the upper portion 104. The lower portion 106 can received the feet and crossed legs of the user.

FIG. 5 is a rear elevation view of the vibroacoustic stimulation delivery system of FIG. 3.

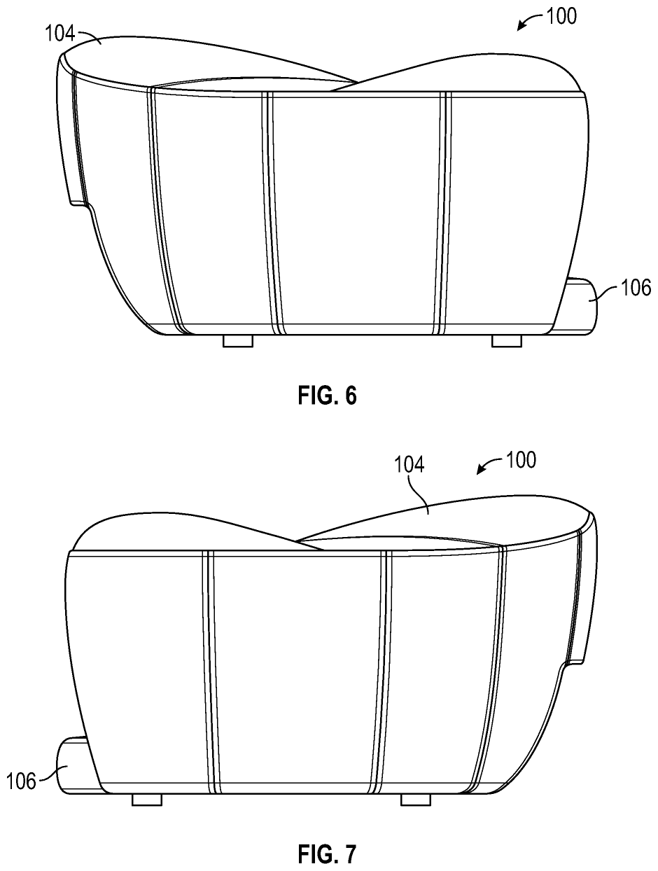

FIG. 6 is a right-side elevation view of the vibroacoustic stimulation delivery system of FIG. 3.

FIG. 7 is a left-side elevation view of the vibroacoustic stimulation delivery system of FIG. 3.

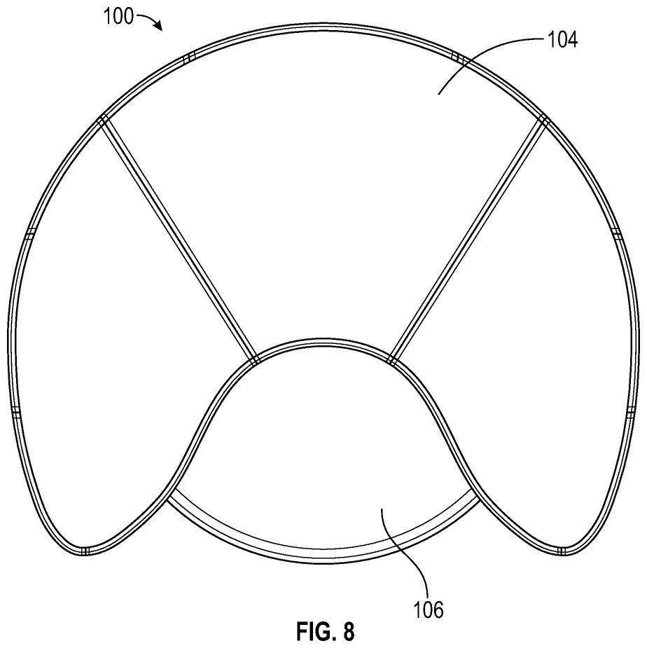

FIG. 8 is a plan view of the vibroacoustic stimulation delivery system of FIG. 3. The outer shape of the seating apparatus 102 can be circular, oval (e.g., FIG. 1), square, triangular, or any other shape as needed.

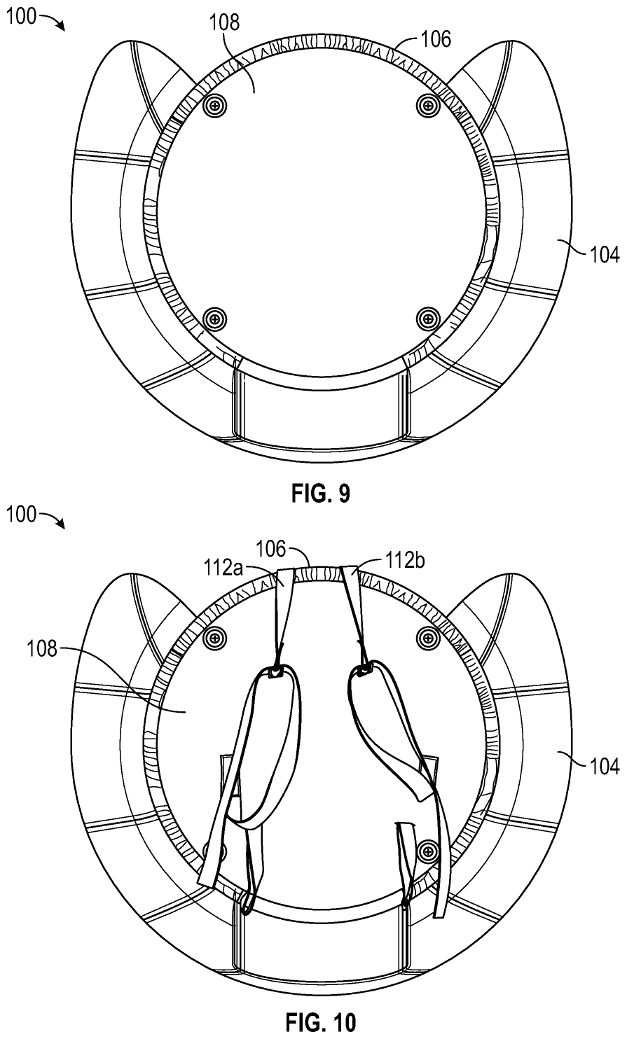

FIG. 9 is a bottom view of an embodiment of the vibroacoustic stimulation delivery system of FIG. 1. The system 100 can have a removable bottom panel 108 that can be removed to access the internal components of the system 100. For example, the bottom panel can be secured by a zipper or hook-and-loop fasteners to conceal the audio subsystem 230. The bottom panel 108 can further be removed as needed to service or replace various components of the control system 200.

FIG. 10 is a bottom view of another embodiment of the vibroacoustic stimulation delivery system of FIG. 9. Carrying straps 112 (shown as carrying straps 112a, 112b) can be coupled to the seating apparatus 102. In some embodiments the carrying straps 112 can be formed similar to backpack straps as shown for easy carry. The carrying straps 112 may be detachable or otherwise removable. The seating apparatus 102 can further have pockets for stowing the straps or personal items. The pockets for stowage can be positioned on, for example, the sides or the bottom of the seating apparatus 102 as needed.

FIG. 11 is a graphical representation of another embodiment of the vibroacoustic stimulation delivery system of FIG. 1. A seating apparatus 500 can be similar to the system 100 and the seating apparatus 102 (FIG. 1). The seating apparatus 500 can be formed as a chair 502 and include the control system 200 with the listening devices 110 and the tactile transducers 120 for delivering audio and vibrations throughout the chair 502. The chair 502 can further have an upper portion 504 and a lower portion 506, similar to the seating apparatus 102.

FIG. 12 is a graphical representation of another embodiment of the vibroacoustic stimulation delivery system of FIG. 1. A seating apparatus 600 can be similar to the seating apparatus 102 and formed as a meditation pad or cushion. The seating apparatus 600 can have a single piece, round padded construction having similar internal components as those described above in connection with the vibroacoustic stimulation delivery system 100 and the control system 200.

FIG. 13 is a graphical representation of another embodiment of the vibroacoustic stimulation delivery system of FIG. 1. A device 700 can be similar to the vibroacoustic stimulation delivery system 100, but formed as a block 702. In some examples, the block 702 can be a yoga block. Accordingly, the device 700 can include the components of the system 100, including the tactile transducers 120 and the control system 200 within the body of the block 702. The listening devices 110 can also be included. In some implementations, the device 700 can have one or more speaker ports 710 in the block 702 to for audio from internal listening devices 110. In some implementations, the device 700 may omit the speaker ports 710 and instead include wireless headphones as the listening devices 110. In some implementations, the block 702 can have an access port 712, providing access to the internal components of the vibroacoustic stimulation delivery system 100 or the control system 200 on the interior of the block 702.

The block 702 can have a rigid body having rectangular prism shape. The rigid body can have a soft exterior (e.g., rubberized) coating similar to a yoga block as is known in the art. The block 702 can have the audio input 222 configured to receive an audio signal. The audio signal can be delivered in a wireless manner (e.g., WiFi, Bluetooth, etc.) over a first wireless link. The block 702 can contain at least one tactile transducer 120 disposed within the rigid body and configured to product vibrations according to low frequency components of the audio signal. The block 702 can have a listening device configured to output an audible version of the audio signal synchronized with the vibrations. The audio signal can be delivered to the listening device 110 via a second wireless link. The listening device 110 in this example can be wireless headphones. In some embodiments, the first wireless link can be different from the second wireless link to comport with Bluetooth, WiFi or other applicable standards or protocols.

Those of skill will appreciate that the various illustrative logical blocks (e.g., the various servers described herein) described in connection with the embodiments disclosed herein can often be implemented as electronic hardware, computer software, or combinations of both. To clearly illustrate this interchangeability of hardware various illustrative components, blocks, and modules have been described above generally in terms of their functionality. Whether such functionality is implemented as hardware or software depends upon the design constraints imposed on the overall system. Skilled persons can implement the described functionality in varying ways for each particular application, but such implementation decisions should not be interpreted as causing a departure from the scope of the disclosure. In addition, the grouping of functions within a module, block or step is for ease of description. Specific functions or steps can be moved from one module or block without departing from the disclosure.

It will be understood that the benefits and advantages described above may relate to one embodiment or may relate to several embodiments. The embodiments are not limited to those that solve any or all of the stated problems or those that have any or all of the stated benefits and advantages.

Any reference to ‘an’ item refers to one or more of those items. The term ‘comprising’ is used herein to mean including the method blocks or elements identified, but that such blocks or elements do not comprise an exclusive list and a method or apparatus may contain additional blocks or elements.

What is claimed is:

1. A system for delivering vibroacoustic stimulation, comprising:
a seating apparatus;
an acoustic subsystem contained within the seating apparatus having an audio input configured to receive an audio signal;
one or more tactile transducers contained within the seating apparatus and communicatively coupled to the acoustic subsystem and configured to provide vibrations according to low frequency elements of the audio signal;
a listening device coupled to the acoustic subsystem and configured to produce an audio signal to a user in conjunction with the vibrations; and
a user interface configured to receive user input regarding the audio signal.

2. The system of claim 1, wherein the seating apparatus comprises a meditation mat.

3. The system of claim 1, wherein the seating apparatus comprises a meditation cushion having an upper portion and a lower portion.

4. The system of claim 3, wherein the lower portion has a first diameter and the upper portion has a second diameter larger than the first diameter by the Golden Ratio (1.61803398875).

5. The system of claim 1, further comprising a delay unit communicatively coupled to the audio input and configured to impart a delay on the audio signal.

6. The system of claim 1, wherein the listening device comprises one or more speakers contained within the seating apparatus.

7. The system of claim 1, wherein the listening device comprises headphones worn by the user.

8. The system of claim 1, wherein the listening device comprises one or more speakers contained within the seating apparatus and headphones.

9. A device for enhanced meditation experience comprising:
a meditation cushion having an upper portion for receiving a rear of a user and a lower portion for receiving feet and legs of the user;
an audio input configured to receive an audio signal;
one or more tactile transducers contained within the meditation cushion and communicatively coupled to the acoustic subsystem, the one or more tactile transducers being configured to provide vibrations according to low frequency elements of the audio signal;
a listening device coupled to the acoustic subsystem and configured to produce an audio signal to a user in conjunction with the vibrations; and
a user interface configured to receive user input regarding the audio signal.

10. The device of claim 9, wherein the lower portion has a first diameter and the upper portion has a second diameter larger than the first diameter by the Golden Ratio (1.61803398875).

11. The device of claim 9, further comprising a delay unit communicatively coupled to the audio input and configured to impart a delay on the audio signal.

12. The device of claim 9, wherein the listening device comprises one or more speakers contained within the meditation cushion.

13. The device of claim 12, wherein the one or more speakers are embedded within the upper portion of the meditation cushion.

14. The device of claim 9, wherein the one or more tactile transducers are embedded within the upper portion of the meditation cushion.

15. A device providing vibroacoustic stimulation comprising:
a rigid body, having rectangular prism shape;
an audio input configured to receive an audio signal;
at least one tactile transducer disposed within the rigid body and configured to product vibrations according to low frequency components of the audio signal; and
a listening device configured to output an audible version of the audio signal synchronized with the vibrations.

16. The device of claim 15, wherein the rigid body comprises a block.

17. The device of claim 15, wherein the audio input comprises a first wireless link.

18. The device of claim 15, wherein the audio output comprises a second wireless link.
